(19) United States
(12) Patent Application Publication
(10) Pub. No.: US 2023/0285284 A1
(43) Pub. Date: Sep. 14, 2023

(54) LUBRICANT PODS

(71) Applicant: Elizabeth McQuaid, Sacramento, CA (US)

(72) Inventor: Elizabeth McQuaid, Sacramento, CA (US)

(21) Appl. No.: 18/181,405

(22) Filed: Mar. 9, 2023

Related U.S. Application Data
(60) Provisional application No. 63/318,681, filed on Mar. 10, 2022.

(51) Int. Cl. A61K 9/02; A61K 9/00

(52) U.S. Cl. CPC A61K 9/025; A61K 9/0031; A61K 9/0036

(57) ABSTRACT

A lubricant container configured to facilitate the delivery of lubricant steadily, over time, within a target cavity such as a vagina or anus is described. The container is composed of a soluble membrane or substrate configured to dissolve at or above body temperature. Subdivisions within the container, preferably established via dividers, are configured to contain differing types of lubricant which are to be released in differing stages after a certain amount of time has elapsed. Differing portions of the container, as well as the dividers, are preferably composed of substrate having differing densities and/or thicknesses to facilitate the time-delay function of the apparatus.

This application is a non-provisional patent application of provisional patent application No. 63/318,681, filed on Mar. 10, 2022, and priority is claimed thereto.

### FIELD OF THE PRESENT INVENTION

The present invention relates to the field of personal use intercourse aids, and more specifically relates to a dissolvable membrane, exhibiting a shape similar to a suppository or pill, configured to contain personal lubricant for disposition within a vagina or anus of a user.

### BACKGROUND OF THE PRESENT INVENTION

Many post-menopause women experience vaginal dryness. As a result, sexual intercourse becomes painful. In such scenarios, lubricant, colloquially referenced as “lube” is the preferred mechanism by which the pain can be reduced or eliminated.

Unfortunately, traditional lubrication delivery methods of squirting lube from a bottle onto the hand, and manually applying the lube to sexual areas is messy. In general, it can kill the mood. Additionally, lube must be reapplied frequently to achieve the desired effect. This can often lead to excess lube on the hands getting onto sheets or surrounding furniture.

If there were a way in which users could achieve the benefits of the use of lube without the consequence of messy hands and sheets, users would feel more comfortable using such lubricants more often, and therefore more individuals would have a more pleasant experience with lube. Additionally, if there were a way in which lube could be provided in a time-delayed manner, the need to reapply lube could be minimized or eliminated.

Thus, there is a need for an apparatus configured to deliver lubricant, without a mess, within the vagina or anus directly prior to the beginning of intercourse. Such an apparatus preferably employs a glycerin-based, dissolvable membrane encompassing a predetermined amount of lubricant. The membrane is configured to melt at or slightly below body temperature via a time-release function, releasing the lubricant contained therein within the vagina or anus, thereby lubricating the vagina or anus.

### SUMMARY OF THE PRESENT INVENTION

The present invention is a lubricant container equipped with a dissolvable, non-toxic membrane, configured to release lubricant in a delayed fashion upon exposure to body temperature for an extended period of time. The entirety of the container is composed of the membrane. The thickness of the membrane may vary to facilitate a time-delayed release. Interior walls within the container may be present, exhibiting differing thicknesses of membrane to ensure thicker membranes dissolve more slowly than others to achieve the time-delayed feature of the apparatus. The container is smooth and is configured to be disposed within the vagina or anus of a user to facilitate lubrication of the vagina or anus prior to intercourse.

The following brief and detailed descriptions of the drawings are provided to explain possible embodiments of the present invention but are not provided to limit the scope of the present invention as expressed herein this summary section.

### BRIEF DESCRIPTION OF THE DRAWINGS

The accompanying drawings, which are incorporated herein and form a part of the specification, illustrate the present invention and, together with the description, further serve to explain the principles of the invention and to enable a person skilled in the pertinent art to make and use the invention.

The present invention will be better understood with reference to the appended drawing sheets, wherein:

FIG. 1 depicts a view of the dissolvable lubricant container of the present invention as seen from the side, detailing interior walls (dividers) of the container composed of the non-toxic dissolvable membrane.

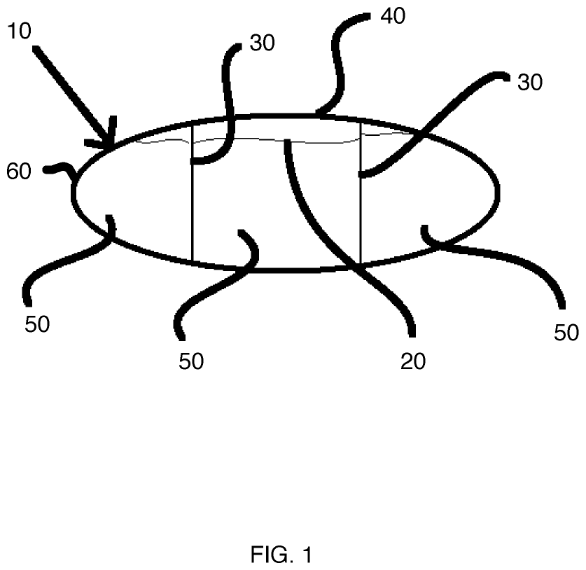

FIG. 2 shows a perspective view of the dissolvable lubricant container of the present invention.

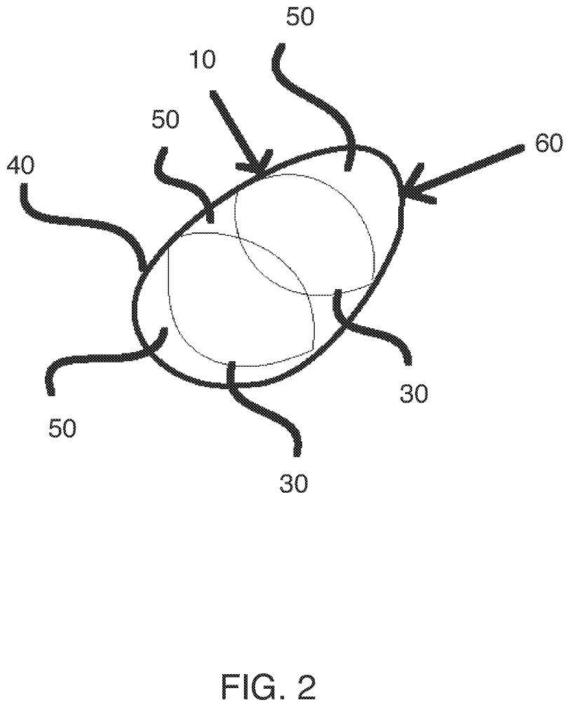

FIG. 3 exhibits a flow chart detailing the process of use of the apparatus of the present invention by a user.

### DETAILED DESCRIPTION OF THE PREFERRED EMBODIMENT

The present specification discloses one or more embodiments that incorporate the features of the invention. The disclosed embodiment(s) merely exemplify the invention. The scope of the invention is not limited to the disclosed embodiment(s).

References in the specification to “one embodiment,” “an embodiment,” “an example embodiment,” etc., indicate that the embodiment described may include a particular feature, structure, or characteristic, but every embodiment may not necessarily include the particular feature, structure or characteristic. Moreover, such phrases are not necessarily referring to the same embodiment, Further, when a particular feature, structure, or characteristic is described in connection with an embodiment, it is submitted that it is within the knowledge of one skilled in the art to effect such feature, structure, or characteristic in connection with other embodiments whether or not explicitly described.

The present invention is a lubricant delivery apparatus configured to store and contain personal lubricant (20) within a container (10). The container (10) is configured to release the lubricant (20) when the container (10) and/or portions of the container (10) dissolve. The container (10) is preferably composed of a glycol based, non-toxic gelatinous substrate (40) which does not melt at room temperature or general ambient temperature. Instead, the substrate (40), also referenced as a membrane, only dissolves upon sustained contact with human body temperature. As such, the container (10) of the present invention is configured to only release the lubricant (20) after it has been placed in the desired location for the use of said lubricant (20), namely within the vagina or the anus.

The container (10) is preferably divided via dividers (30) which can segment the container (10) into up to three subdivisions (50). The dividers (30) are composed of the same substrate (40) or membrane as that of the container (10) itself. However, the thickness of the substrate (40) varies for each divider (30) and subdivision (50). Each subdivision (50) is preferably surrounded by rounded or curved walls (60) of the container (10) and is therefore encompassed by the substrate (40). It is envisioned that there are three different thicknesses or densities of substrate (40), each configured to dissolve at a different rate due to their composition.

Each subdivision (50) of the container (10) may contain differing types of lubricant (20) as well. For example, a first subdivision (50) may contain traditional lubricant, a second subdivision (50) may contain a warming or cooling lubricant (conventionally comprised), and a third subdivision (50) may contain a soothing lubricant. The solubility and viscosity of the lubricant in each subdivision (50) of the container (10) may differ as well in accordance with user preference. Similarly, lubricant (20) contained in some or all of the subdivisions (50) of the container (10) of the present invention may be flavored by one or more of a variety of known non-toxic flavorings.

Other embodiments of the present invention may be configured for oral use. In such instances, a semi-soluble vitamin solution may be present within the container (10) in order to facilitate a time-delayed dosage of requisite supplements as needed for conventional bodily function. It is envisioned that these embodiments of the container (10) of the present invention be smaller, and are therefore easy to swallow whole. Dosages of the supplements contained therein are precisely measured and monitored to be in compliance with local and federal laws.

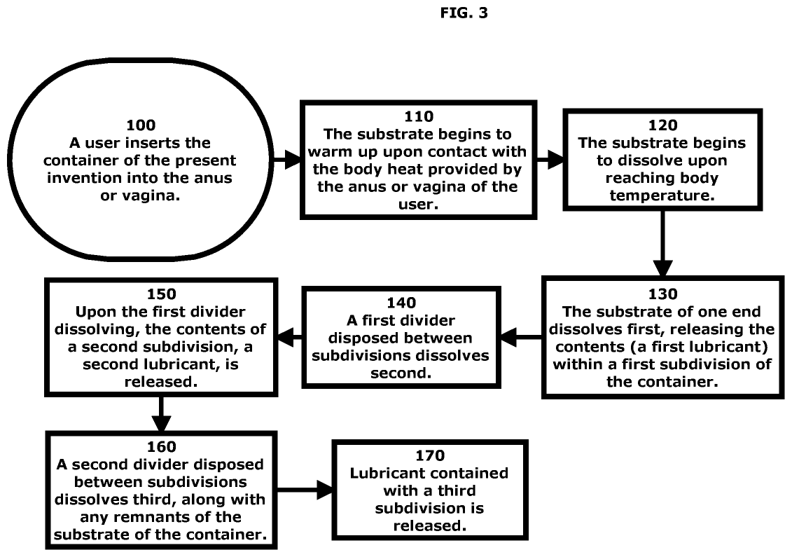

The process of use of the present invention, as detailed in FIG. 3, is preferably as follows:
1. A user inserts the container of the present invention into the anus or vagina. (100)
2. The substrate begins to warm up upon contact with the body heat provided by the anus or vagina of the user. (110)
3. The substrate begins to dissolve upon reaching body temperature. (120)
4. The substrate of one end dissolves first, releasing the contents (a first lubricant) within a first subdivision of the container. (130)
5. A first divider disposed between subdivisions dissolves second. (140)
6. Upon the first divider dissolving, the contents of a second subdivision, a second lubricant, is released. (150)
7. A second divider disposed between subdivisions dissolves third, along with any remnants of the substrate of the container. (160)
8. Lubricant contained with a third subdivision is released. (170)

It should be understood that additional subdivisions within the container (10) of the present invention may be present in some embodiments of the present invention, and the design of the present invention is not limited to two or three subdivisions.

Further, it should be noted that the present invention is configured to facilitate the restoration of a more natural experience and sensation during sexual intercourse. Employment of the lubricant delivery device of the present invention is therefore superior to conventional means of applying lubricant prior to, and during intercourse.

Having illustrated the present invention, it should be understood that various adjustments and versions might be implemented without venturing away from the essence of the present invention. Further, it should be understood that the present invention is not solely limited to the invention as described in the embodiments above, but further comprises any and all embodiments within the scope of this application.

The foregoing descriptions of specific embodiments of the present invention have been presented for purposes of illustration and description. They are not intended to be exhaustive or to limit the present invention to the precise forms disclosed, and obviously many modifications and variations are possible in light of the above teaching. The exemplary embodiment was chosen and described in order to best explain the principles of the present invention and its practical application, to thereby enable others skilled in the art to best utilize the present invention and various embodiments with various modifications as are suited to the particular use contemplated.

I claim:

1. A personal lubricant delivery apparatus comprising:
a container, said container having a body composed of a dissolvable membrane;
wherein said body is three dimensional;
wherein said body exhibits an elongated oval shape;
a first divider, said first divider present within said body;
a second divider, said second divider present within said body;
wherein said first divider and said second divider segment said body into a first segment, a second segment, and a third segment;
wherein said first segment is present at a top end of said body;
wherein said first segment of said body contains a first substance;
wherein said second segment is present within a center of said body;
wherein said second segment contains a second substance;
wherein said third segment is present at a bottom end of said body; and
wherein said third segment contains a third substance.

2. The apparatus of claim 1, wherein said first divider differs in thickness from said second divider; and
wherein said first divider dissolves at a slower rate than said second divider which is thinner.

3. The apparatus of claim 1, wherein said first segment dissolves to release said first substance before said second segment dissolves to release said second substance.

4. The apparatus of claim 1, wherein said second segment dissolves to release said second substance before said third segment dissolves to release said third substance.

5. The apparatus of claim 1, wherein said first substance is a lubricant.

6. The apparatus of claim 1, wherein said second substance is a soothing lubricant exhibiting a solubility and viscosity that differs from that of said first substance.

7. The apparatus of claim 1, wherein said third substance is a warming lubricant.

8. The apparatus of claim 1, wherein said third substance is a cooling lubricant.

9. The apparatus of claim 2, wherein said first segment dissolves to release said first substance before said second segment dissolves to release said second substance.

10. The apparatus of claim 2, wherein said second segment dissolves to release said second substance before said third segment dissolves to release said third substance.

11. The apparatus of claim 2, wherein said first substance is a lubricant.

12. The apparatus of claim 2, wherein said second substance is a soothing lubricant exhibiting a solubility and viscosity that differs from that of said first substance.

13. The apparatus of claim 2, wherein said third substance is a warming lubricant.

14. A personal lubricant delivery apparatus comprising:
a container, said container having a body composed of a dissolvable membrane;
wherein said body is three dimensional;
wherein said body exhibits an elongated oval shape;
a first divider, said first divider present within said body;
a second divider, said second divider present within said body;
wherein said first divider and said second divider segment said body into a first segment, a second segment, and a third segment;
wherein said first segment is present at a top end of said body;
wherein said first segment of said body contains a first substance;
wherein said second segment is present within a center of said body;
wherein said second segment contains a second substance;
wherein said third segment is present at a bottom end of said body;
wherein said third segment contains a third substance;
wherein said first divider differs in thickness from said second divider;
wherein said first divider dissolves at a slower rate than said second divider which is thinner;
wherein said first segment dissolves to release said first substance before said second segment dissolves to release said second substance; and
wherein said first substance, said second substance, and said third substance are personal water-based lubricants that vary in viscosity.
